(19) United States
(12) Patent Application Publication
(10) Pub. No.: US 2008/0200755 A1
(43) Pub. Date: Aug. 21, 2008

(54) Method and device for retrieving suture tags

(76) Inventor: Gregory J. Bakos, Mason, OH (US)

Correspondence Address: KIRKPATRICK & LOCKHART PRESTON GATES ELLIS LLP, 535 SMITHFIELD STREET, PITTSBURGH, PA, 15222, US

(21) Appl. No.: 11/706,811

(22) Filed: Feb. 15, 2007

(51) Int. Cl. A61B 1/00

(52) U.S. Cl. 600/104; 606/138

(57) ABSTRACT

A suture tag retrieval device and method for its use are described. The retrieval device has an elongate member dimensioned in cross-section to allow passage thereof through a channel leading to an internal site in a patient, such as, for example, the working channel of an endoscope. The retrieval device has a distal end and a proximal end and defines a passageway between the distal and the proximal ends for passage of a suture. The distal end of the elongate member has a suture engagement portion and a suture tag receiving portion, which may be in the form of a cradle configured to inhibit contact between the suture tag and the channel. To facilitate use with an endoscope, the elongate member is preferably a hollow, flexible tube like device.

### FIELD OF THE INVENTION

The invention relates to medical instruments and procedures, and more particularly to adjuncts to suturing instruments.

### BACKGROUND

Physicians have often used endoscopes to examine, to biopsy, and to ablate the tissue of patients within lumens such as the esophageous and the bowel or other body cavity and internal patient sites. An endoscope generally includes either a rigid or flexible tube containing one or more optical fiber systems and, for operative uses (human or veterinary), one or more channels for passage of medical instruments. The optical system includes a light delivery system to illuminate the organ or site under inspection and a camera system to transmit the image of the site of interest to the practitioner. Light is typically directed from a source outside the patent by optical fiber bundles in the endoscope to the area of interest.

Endoscopes enable minimally invasive surgical and diagnostic techniques. They are useful in examination and treatment of the gastrointestinal tract, respiratory tract, urinary tract, female reproductive organs, and normally closed body cavities for which specialized instruments, such as the laparoscope for access to the abdominal cavity or the pelvic cavity, the arthroscope for access to the interior of joints, and other site specific instruments for access, for example, to the organs of the chest or during pregnancy, to the amniotic sac or the fetus, are becoming the norm.

More recently, a surgical technique known as natural orifice transenteric surgery (NOTES) is attracting interest. NOTES, which enables “scarless” abdominal operations, may be performed with an endoscope that is passed through a natural orifice (mouth, nose, anus, etc.), then through an internal incision in the stomach or colon, for example, thus avoiding any external incisions or scars. The NOTES technique has been used for diagnostic and therapeutic procedures in animal models, including transgastric (through the stomach) organ removal. Transcolonic approaches are also advocated for access to upper abdominal structures that may be more difficult to work with using a transgastric approach.

A physician performing a therapeutic procedure with the use of an endoscope places a long, flexible instrument through the endoscope's instrument channel and then positions the instrument near the site within the body lumen where a procedure is to be performed. The instrument channels and optical fiber bundles open into the body at the distal end of the endoscope and are generally parallel to the axis of the flexible endoscope. Physicians place flexible instruments through the instrument channels while visualizing and illuminating an internal site using the optical fiber bundles.

Sutures are used to approximate, or bring together, tissue separated, for example, by some trauma, or wound or during a surgical procedure to close an incision or an organ perforation. Suturing instruments generally include a needle and a trailing length of suture material. In some cases, the leading, or distal end, of the suture material is attached to a small tag to stabilize the tissue and the suture as the surgeon pulls the suture material through tissue. In endoscopic procedures, the instruments placed through an instrument channel may include needles and sutures for stitching such a wound, incision or perforation within the patient internal site. An exemplary suturing device is shown in U.S. Pat. No. 7,131,978.

At times, a suture tag misfires; that is, the tag either does not attach to the tissue as intended when initially deployed or the tag and the suture to which it is attached work free of the tissue. Such misfired tags may be retrieved from the patient's body lumen, but, with difficulty. Retrieval heretofore has been achieved by pulling the suture though the working channel of an endoscope, resulting frequently in damage to the channel because the tag tends to assume an orientation transverse to the longitudinal axis of the working channel of the endoscope, scratching or piercing it as the tag is pulled proximally through the channel. Alternatively, the misfired tags and sutures may be left in the body. The strand of suture remaining in the working channel of the endoscope is pushed into the body cavity to clear the channel for a new suture and tag. Although the tag and suture are made of biocompatible material or in some instances, bioabsorbable material, leaving the tags and sutures in the patient is not the optimal option and can create a tangled nest of suture material that the surgeon must work around.

### SUMMARY OF THE INVENTION

The problem presented by the presence of misfired suture tags and sutures in the internal sites of a patient undergoing a procedure is overcome by the suture tag retrieval device of the present invention. The suture tag retrieval device described herein comprises an elongate member dimensioned in cross-section to allow passage thereof through a channel leading to an internal site in a patient, such as, for example, the working channel of an endoscope. The retrieval device has a distal end and a proximal end and defines a passageway between the distal and the proximal ends. The passageway is dimensioned in cross-section to allow passage of a suture therethrough. The distal end of the elongate member has a suture engagement portion and a suture tag receiving portion configured to inhibit contact between the suture tag and the channel. The elongate member may be a hollow, preferably flexible, tube.

In one embodiment, the suture tag receiving portion is configured to align the suture tag with the longitudinal axis of the elongate member. In another embodiment, the suture tag receiving portion may comprise a cradle configured to receive a suture tag in a predetermined orientation. Alternatively, the tag receiving portion may have a surface contoured for engagement with complementary contours of at least a portion of the suture tag.

The suture engagement portion may comprise at least one secondary opening in the elongate member dimensioned for passage of the suture material. The secondary opening may be a hole in suture tag receiving portion of the elongate member. Two or more such openings may be provided. Alternatively, suture guides or brackets may be provided to engage the suture.

A kit may be provided which comprises a suture tag retrieval device. The kit may further include one or more sets of sutures, each suture having a suture tag attached to one end thereof, and suturing instruments. The kit may be provided with an endoscope or may be provided separately as an accessory for use with a previously acquired endoscope.

A method is also provided for retrieving a suture tag that is not attached to tissue from an internal site in a patient wherein the suture tag is attached to a distal end of a suture. The method comprises attaching a proximal end of a suture to a suture engagement portion of a suture tag retrieval device, the retrieval device comprising an elongate member having a distal end and a proximal end, and defining a passageway between the distal and proximal ends, the distal end of the elongate member having the suture engagement portion and a suture tag receiving portion. Following attachment of the suture to the suture engagement portion, the distal end of the suture tag retrieval device is inserted into the proximal end of a channel leading to an internal site in a patient. The method further includes directing the distal end of the suture tag retrieval device through the channel along the length of the suture to a desired location, for example, to the distal end of the channel, to the patient internal site or to a location near the patient internal site. Either while, or after, positioning the distal end of the suture tag retrieval device at the desired location, the proximal end of the suture is pulled proximally to draw a suture tag attached to the distal end of the suture into the suture tag receiving portion of the suture tag retrieval device, and the suture tag retrieval device is withdrawn with the suture tag proximally through the channel.

A method is also provided which includes obtaining a surgical instrument, wherein the surgical instrument comprises a suture tag retrieval device, sterilizing the surgical instrument; and storing the surgical instrument in a sterile container.

### BRIEF DESCRIPTION OF THE DRAWINGS

The novel features of the invention are set forth with particularity in the appended claims. The invention itself, however, both as to organization and methods of operation, together with further objects and advantages thereof, may best be understood by reference to the following description, taken in conjunction with the accompanying drawings in which:

FIG. 1 is a side view of a representative endoscope.

FIG. 2 is a view of the proximal end of the endoscope of FIG. 1 showing the proximal end of a length of suture extending from the endoscope.

FIG. 3 is a perspective view of the distal end of the endoscope showing the extraction device extending from a working channel of the endoscope and a suture tag at the distal end of a length of suture to be retrieved with the extraction device.

FIG. 4 is a side section view of the extraction device through the distal end of an endoscope in the process of retrieving a suture tag.

FIG. 5 is a side section view of the extraction device of FIG. 4 showing the suture tag nesting in a cradle in the extraction device.

FIG. 6 is a top view of the extraction device.

FIG. 7 is a bottom view of the extraction device with a section of suture looped through holes in the cradle portion.

FIG. 8 is a view of the distal end of the extraction device showing the suture tag being pulled toward the extraction device.

FIG. 9 is a view of the distal end of the extraction device showing the suture tag nested in the cradle portion of the extraction device.

### DETAILED DESCRIPTION

Before the present method and embodiments of an instrument are disclosed and described, it is to be understood that this invention is not limited to the particular process steps and materials disclosed herein as such process steps and materials may vary somewhat. It is also to be understood that the terminology used herein is used for the purpose of describing particular embodiments only and is not intended to be limiting since the scope of the present invention will be limited only by the appended claims.

Unless defined otherwise, all technical and scientific terms used herein have the same meaning as commonly understood to one of ordinary skill in the art to which this invention belongs. Although any method, instrument and materials similar or equivalent to those described herein may be used in the practice or testing of the invention, particular embodiments of a method, instrument and materials are now described.

It must be noted that, as used in this specification and the appended claims, the singular forms “a,” “an,” and “the” include plural referents unless the content clearly dictates otherwise.

In describing and claiming the present invention, the following terminology will be used in accordance with the definitions set out below.

As used herein, the term “patient,” used herein, refers to any human or animal on which an endoscopic procedure may be performed.

As used herein, the term “biocompatible” includes any material that is compatible with the living tissues and system(s) of a patient by not being substantially toxic or injurious and not causing immunological rejection. “Biocompatibility” includes the tendency of a material to be biocompatible.

As used herein, the term “bioabsorbable” includes the ability of a material to be dissolved and/or degraded, and absorbed, by the body.

As used herein, the term “proximal” (or any form thereof), with respect to a component of an instrument, means that portion of the component that is generally nearest the surgeon, or nearest to the end of the instrument handled by the surgeon, when in use; and with respect to a direction of travel of a component of an instrument, means toward the end of the instrument generally nearest the surgeon, or handled by the surgeon, when in use.

As used herein, the term “distal” (or any form thereof), with respect to a component of an instrument, means that portion of the component that is generally farthest from the surgeon, or farthest from the end of the instrument handled by the surgeon, when in use; and with respect to a direction of travel of a component of an instrument, means away from the end of the instrument generally nearest the surgeon, or handled by the surgeon, when in use.

As used herein, the term “transverse” (or any form thereof), with respect to an axis, means extending in a line, plane or direction that is across such axis, i.e., not collinear or parallel therewith. “Transverse” as used herein is not to be limited to “perpendicular”.

As used herein, the term “longitudinal axis”, with respect to an instrument, means the exact or approximate central axis defined by said instrument along its greater dimension, i.e., along its length, from its distal end to its proximal end, and vice versa, and is not intended to be limited to imply a straight line, wherein, for example, an instrument includes a bend angle or curve, it is intended that “longitudinal axis” as used herein follows such bend angle or curve.

As used herein, the term “internal site” of a patient means a lumen, body cavity, internal organ or other location in a patient's body including, without limitation, sites accessible through natural orifices or through incisions or both.

The present invention has application in conventional endoscopic and open surgical instrumentation, as well as application in robotic-assisted surgery. The embodiments shown illustrate the use of the invention in connection with an endoscope within an internal site of a patient. The invention is useful in a variety of minimally invasive medical procedures, including without limitation medical procedures performed through laparoscopic incisions for access to body cavities and internal organs of the body. The invention also encompasses apparatus and methods employing endoscopic devices in general, including various forms and variations of endoscopes, including without limitation: laparoscopes, gastroscopes, peritoneoscopes, sigmoidoscopes, fiberoptic endoscopes, arthroscopes, amnioscopes, and the like.

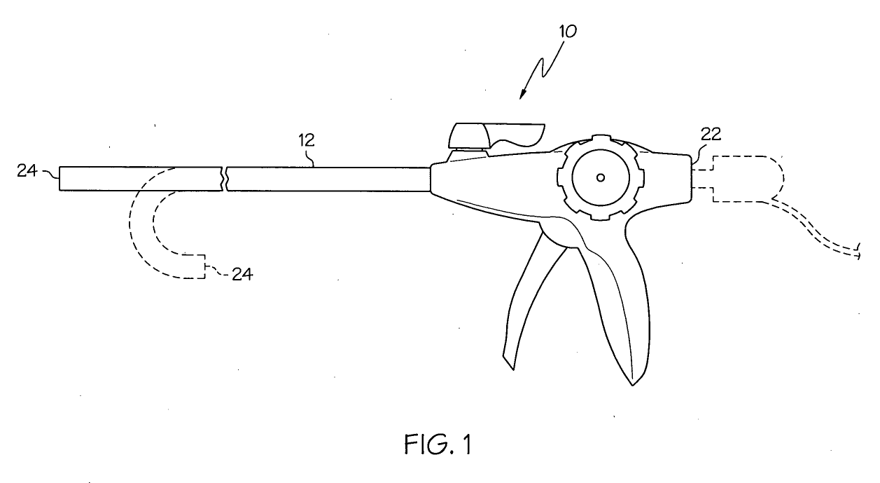

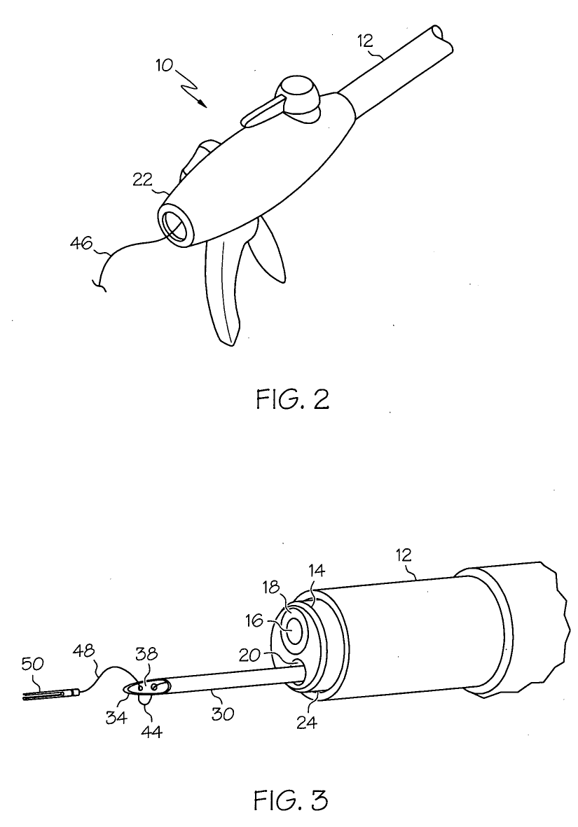

Referring to FIGS. 1 and 2, a representative endoscope 10 includes generally a proximal end 22 and distal end 24, an insertion tube 14 and a sleeve 12 covering a major portion of the insertion tube 14. At the proximal end 22, hand pieces and controls are provided for use by a practitioner, such as a clinician, physician or surgeon. The insertion tube 14 is preferably removable for cleaning and both the insertion tube and the sleeve are flexible for at least a major part of their lengths. In use, the sleeve 12 and insertion tube 14 lead to an internal site of interest in a patient. At the distal end 24 of the insertion tube 14, a camera 16 and lights 18 are provided to enable the practitioner to see the internal site of the patient. The insertion tube 14 also includes one or more working channels 20 (one is shown) through which various instruments are typically inserted to allow the practitioner to perform desired procedures at the internal site. The working channel 20 includes a longitudinal axis 28, as defined herein.

One such procedure requires suturing (or stitching) a wound, incision or other perforation within the patient. As described above, a suturing instrument (not shown) would be inserted through the working channel 20 of the endoscope 10. A length of suture 44 having a proximal end 46 and a distal end 48, with a suture tag 50 attached by suitable known means to the distal end 48 of the suture 44 would typically be inserted through the working channel 20. In one method of suturing, the suture tag 50 is secured to one side of the patient tissue to be sutured and a desired length of the suture 44 trailing the suture tag 50 is stitched through the tissue to close the wound, incision or perforation. From time to time, the suture tag 50 and suture 44 either do not attach to the tissue as intended or become loose and pull away from the tissue as the practitioner stitches the tissue. When that happens, the proximal end 46 of the suture 44 may remain in the working channel 20 of the endoscope 10 or may be pulled into the working channel 20 with a suitable instrument.

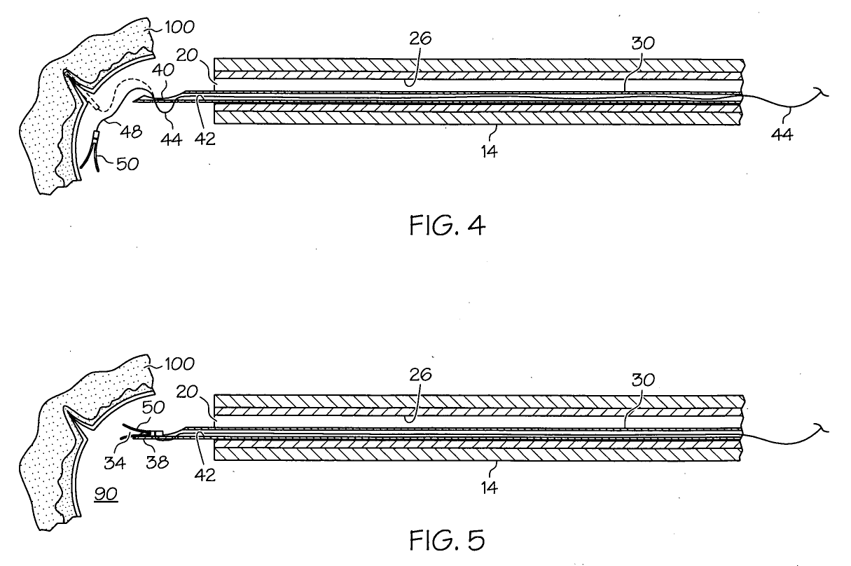

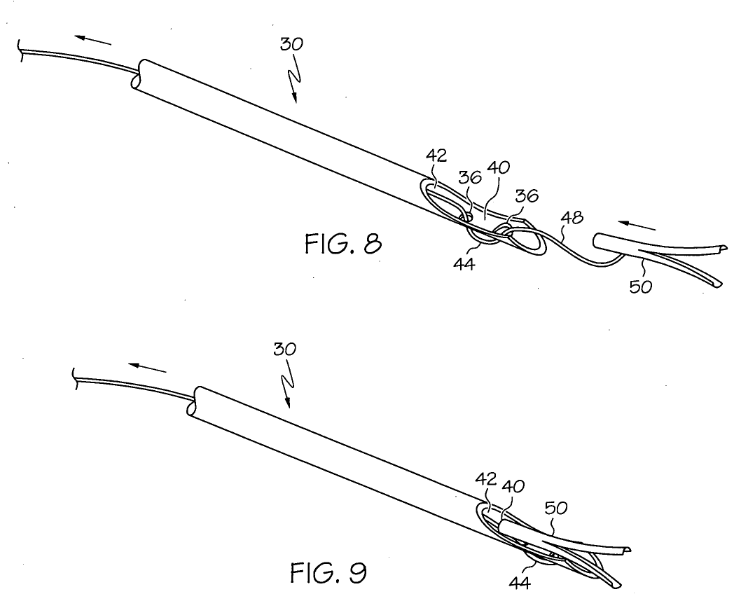

The suture tag retrieval device 30 shown in FIGS. 3-9, is provided to allow extraction of the loose or misfired suture tag 50 and suture 44 from the patient internal site through the channel 20 without damaging the channel 20. The suture tag retrieval device 30 includes an elongate member having a passageway 42, a longitudinal axis 52, a proximal end 32 and a distal end 34. The retrieval device 30 may be made of any suitable biocompatible material and in the embodiment shown, is suitably flexible for use within the working channel 20 of an endoscope. The degree of flexibility may vary depending on the type of endoscope and the length of the channel 20 through which the retrieval device 30 must travel.

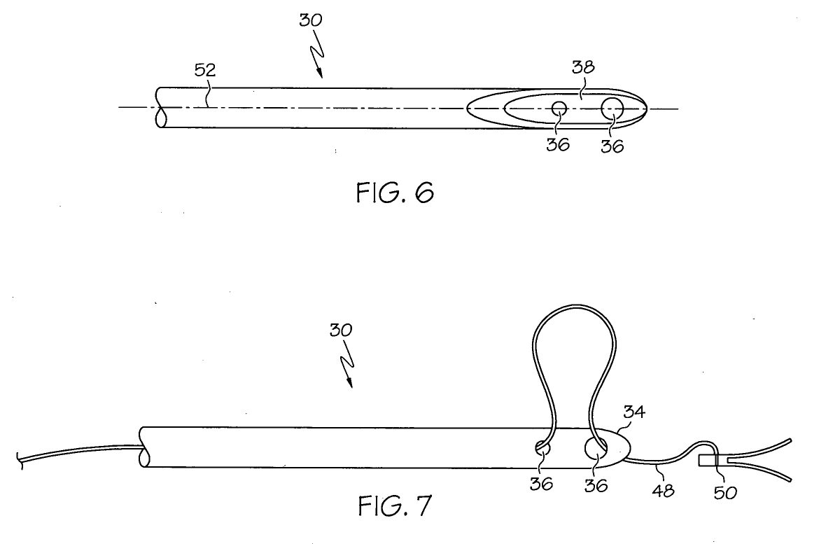

The retrieval device 30 also includes a suture engagement portion 36, which may be positioned along the length of the elongate member. In the embodiment shown, the suture engagement portion 36 is positioned at the distal end 34 of the retrieval device 30. Those skilled in the art will recognize that the suture engagement portion 36 may be positioned some distance proximal to the position shown as long as the proximal end of the suture 46 can be secured to the engagement portion 36 and the engagement of the suture 44 is sufficiently close to the distal end 34 of the retrieval device 30 to guide the suture tag 50, as will be described herein. The suture engagement portion 36, as shown in the embodiment of FIGS. 6-9, forms secondary openings, or holes. Those skilled in the art will recognize that other means of engaging the suture 44 may be provided instead of holes. Guide slots, or guide sleeves, grooves, brackets or the like may be provided to releasably attach the suture to the retrieval device for the extraction of the suture tag 50.

The retrieval device 30 also includes a suture tag receiving portion 38, which may be in the form of a cradle 40 having a trough-like depression for receiving the suture tag 50 in a pre-determined orientation. The orientation should be one that inhibits contact between the suture tag 50 and the interior surfaces 26 of channel 20 when the suture tag 50 is withdrawn through the channel 20, as will be described below. The cradle 40 may include surface contours which complement the surface contours of the suture tag 50 such that when drawn into the cradle 50, the surfaces of the suture tag 50 seat in the complementary surfaces of the cradle 40 to inhibit movement of the suture tag 50 as it is withdrawn through the channel 20.

Alternatively, the surfaces of the cradle 40 may be generally smooth, but the suture tag 50 may be held in place within the depression by the sides of the cradle. The longitudinal axis of the receiving portion 38 is in general alignment with the longitudinal axis 52 of the retrieval device 30, which itself is in general alignment, in use, with the longitudinal axis of the channel 20. When positioned within the receiving portion 38, the longitudinal axis of the tag 50 is aligned with the longitudinal axis of the receiving portion 38 and thereby, with the longitudinal axis 52 of the retrieval device 30.

Referring to FIG. 2, the proximal end 46 of suture 44 is shown extending from the proximal end 22 of an endoscope 10. The proximal end 46 of the suture 44 is attached to the suture engagement portion of the retrieval device 30 by lacing the proximal end 46 of the suture 44 through secondary openings 36 in the distal end 34 of the retrieval device 30. The retrieval device 30 is inserted into the proximal end of the working channel 20 and advanced distally through the channel 20 as the length of suture 44 passes through passageway 42 of retrieval device 30. Those skilled in the art will appreciate that the cross-sectional dimension of the passageway 42 (or the inner diameter of the retrieval device 30, if the passageway is cylindrical) must be large enough to allow passage of suture 44 therethrough. Those skilled in the art will also recognize from the operation of the retrieval device 10 that the cross-sectional dimension (or outer diameter, if the device is cylindrical) of the retrieval device 10 must be less than the inner cross-sectional dimension (or inner diameter, if the channel is cylindrical) of the channel 20 to allow passage of the retrieval device 30 through the channel 20.

The retrieval device 30 is pushed through the channel 20 until it reaches the distal end 24 of the channel 20, or, as shown in FIGS. 3-5, extends distally beyond the end 24 into the body cavity 90 of the patient adjacent tissue 100 where the suture tag 50 is located. The proximal end 46 of the suture 44 is pulled proximally to draw the suture tag 50 towards and into the suture tag receiving portion 38 of retrieval device 30. The suture 44 may be pulled while the retrieval device 30 is being advanced distally along the channel 20 or the practitioner may wait until the retrieval device 30 is in the desired position at the distal end 24 of the channel 20 or just beyond the channel 20 at or near the location of suture tag 50 in body cavity 90.

The suture tag 50 nests in or is cradled in, the receiving portion 38 such that its longitudinal axis is aligned with the longitudinal axis 42 of the retrieval device 30. In this orientation, the suture tag 50 will be inhibited from moving in a transverse direction relative to the longitudinal axis 28 of channel 20 and is inhibited from contact with the interior surfaces 26 of channel 20 as the suture tag 50 is withdrawn through the channel 20.

When the suture tag 50 is positioned in the desired orientation within the suture tag receiving portion 38 of retrieval device 30, the retrieval device 30 is pulled proximally through the channel 20 to remove the suture tag 50 and suture 44 from the patient internal site without damage to the channel 20 of the endoscope 10.

In summary, numerous benefits are apparent which result from employing the concepts of the invention. The foregoing description of one or more embodiments of the invention has been presented for purposes of illustration and description. It is not intended to be exhaustive or to limit the invention to the precise form disclosed. Obvious modifications or variations are possible in light of the above teachings. The embodiments were chosen and described in order to best illustrate the principles of the invention and its practical application to thereby enable one of ordinary skill in the art to best utilize the invention in various embodiments and with various modifications as are suited to the particular use contemplated. It is intended that the scope of the invention be limited only by the claims appended hereto.

The devices disclosed herein can be designed to be disposed of after a single use, or they can be designed to be used multiple times. In either case, however, the device can be reconditioned for reuse after at least one use. Reconditioning can include any combination of the steps of disassembly of the device, followed by cleaning or replacement of particular pieces, and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present invention.

Preferably, the various embodiments of the invention described herein will be processed before patient use. First, a new or used instrument is obtained and if necessary cleaned. The instrument can then be sterilized by any suitable known sterilization technique. This can be done by any number of ways known to those skilled in the art including beta or gamma radiation, ethylene oxide, steam. In one sterilization technique, the instrument is placed in a closed and sealed container, such as a plastic or TYVEK bag. The container and instrument are then placed in a field of radiation that can penetrate the container, such as gamma radiation, x-rays, or high-energy electrons. The radiation kills bacteria on the instrument and in the container. The sterilized instrument can then be stored in the sterile container. The sealed container keeps the instrument sterile until it is opened in the medical facility.

1. A suture tag retrieval device comprising:
an elongate member having a longitudinal axis and being dimensioned in cross-section to allow the elongate member to pass through a channel leading to an internal site in a patient, the elongate member having a distal end and a proximal end and defining a passageway between the distal and proximal ends, the passageway being dimensioned in cross-section to allow passage of a suture therethrough;
the distal end of the elongate member having a suture engagement portion and a suture tag receiving portion configured to inhibit contact between the suture tag and the channel.

2. The suture tag retrieval device recited in claim 1 wherein the suture tag receiving portion at the distal end of the elongate member is configured to generally align the suture tag with the longitudinal axis of the elongate member.

3. The suture tag retrieval device recited in claim 1 wherein the suture tag receiving portion at the distal end of the elongate member comprises a cradle configured to receive a suture tag in a predetermined orientation.

4. The suture tag retrieval device recited in claim 1 wherein the suture engagement portion comprises at least one secondary opening in the elongate member dimensioned for passage of the suture.

5. The suture tag retrieval device recited in claim 4 wherein there are two secondary openings in the elongate member for passage of the suture.

6. The suture tag retrieval device recited in claim 1 wherein the elongate member is flexible.

7. The suture tag retrieval device recited in claim 1 wherein the tag receiving portion has a surface contoured for engagement with complementary contours of at least a portion of the suture tag.

8. The suture tag retrieval device recited in claim 1 wherein the elongate member comprises a hollow tube.

9. The suture tag retrieval device recited in claim 1 wherein the channel is the working channel of an endoscope.

10. In an endoscopic procedure wherein a suture having a proximal end and a distal end is directed through a channel to an internal site of a patient, a method for retrieving a suture tag from the internal site of the patient wherein the suture tag is not attached to tissue and is attached to the distal end of the suture, the method comprising:
attaching the proximal end of the suture to a suture engagement portion of a suture tag retrieval device, the retrieval device comprising an elongate member having a longitudinal axis, a distal end and a proximal end, and defining a passageway between the distal and proximal ends, the distal end of the elongate member having the suture engagement portion and a suture tag receiving portion;
inserting the distal end of the suture tag retrieval device into a proximal end of the channel;
directing the suture tag retrieval device distally through the channel along the length of the suture to a location at or distal to a distal end of the channel;
pulling the proximal end of the suture proximally to draw the suture tag attached to the distal end of the suture into the suture tag receiving portion of the suture tag retrieval device in an orientation such that contact between the suture tag and the channel is inhibited; and
withdrawing the suture tag retrieval device with the suture tag proximally through the channel.

11. The method recited in claim 10 wherein the channel is a working channel of an endoscope.

12. The method recited in claim 10 wherein the suture tag is drawn into the suture tag receiving portion of the elongate member in an orientation generally along the longitudinal axis of the elongate member.

13. A method comprising:
obtaining a surgical instrument, wherein the surgical instrument comprises:
a suture tag retrieval device comprising an elongate member having a distal end and a proximal end, and defining a passageway between the distal and proximal ends, the distal end of the elongate member having a suture engagement portion and a suture tag receiving portion;
sterilizing the surgical instrument; and
storing the surgical instrument in a sterile container.

14. A kit comprising a suture tag retrieval device, said retrieval device comprising an elongate member having a longitudinal axis, a distal end and a proximal end, and defining a passageway between the distal and proximal ends, the distal end of the elongate member having a suture engagement portion and a suture tag receiving portion.

15. The kit recited in claim 14 further comprising a set of sutures, each suture within said set being attached at one end thereof to a suture tag.

16. The kit recited in claim 14 further comprising an endoscopic suturing instrument.

17. The kit recited in claim 14 wherein the suture tag receiving portion at the distal end of the elongate member is configured to align the suture tag generally with the longitudinal axis of the elongate member.

18. The kit recited in claim 14 wherein the suture tag receiving portion at the distal end of the elongate member comprises a cradle configured to receive a suture tag generally in a predetermined orientation.

19. The kit recited in claim 14 wherein the suture engagement portion comprises at least one secondary opening in the elongate member dimensioned for passage of a suture.
